(19) United States
(12) Patent Application Publication
(10) Pub. No.: US 2023/0260619 A1
(43) Pub. Date: Aug. 17, 2023

(54) ALGORITHMIC SERVICE AND TRAINING RECOMMENDATIONS BASED ON DATA, AND ASSOCIATED SYSTEMS AND METHODS

(71) Applicant: Strive Tech Inc., (US)

(72) Inventors: Nikola Mrvaljevic, Bothell, WA (US); Michael Mikhaylov, Bothell, WA (US)

(73) Assignee: Strive Tech Inc., Bothell, WA (US)

(21) Appl. No.: 17/796,034

(22) PCT Filed: Feb. 17, 2022
(86) PCT No.: PCT/US2022/016727

§ 371 Date: Jul. 28, 2022

(51) Int. Cl. G16H 20/30; A63B 24/00; A61B 5/397; A61B 5/11; A61B 5/00

(52) U.S. Cl. CPC G16H 20/30; A63B 24/0075; A63B 24/0062; A61B 5/397; A61B 5/1118; A61B 5/6804; A63B 2220/836; A63B 2225/50; A63B 2230/08; A63B 2230/60; A61B 2503/10

(57) ABSTRACT

Systems and methods for providing algorithmic training and service recommendations are disclosed herein. In one embodiment, a method for treating a fatigue or an injury of an athlete includes: monitoring a first amplitude of a first muscle of the athlete by a first wearable muscle response sensor carried by the athlete; monitoring a second amplitude of a second muscle of the athlete by a second wearable muscle response sensor carried by the athlete; determining a difference between the first amplitude and the second amplitude; comparing the difference to a predetermined amplitude threshold; and based on the comparing, providing a treatment recommendation to the athlete.

### CROSS REFERENCE TO RELATED APPLICATION

This application claims the benefit of U.S. Provisional Application No. 63/151,338 filed Feb. 19, 2021, the disclosure of which is hereby incorporated by reference in its entirety.

### BACKGROUND

It is well known that the athletes, whether professional or otherwise, are subject to injuries. In some cases, such injuries are preceded with signs of fatigue. When a trainer (e.g., an athletic trainer or coach) detects signs of fatigue, the trainer can intervene to reduce the likelihood of fatigue-related injury. For example, when a trainer detects fatigue, the trainer may instruct the athlete to slow down or focus on technique or the trainer may pull the athlete from a game or a practice session for rest and recovery. In addition or alternatively, the trainer may provide recommended exercise that is typically less strenuous than the normal exercise. Such recommendations are typically provided based on the trainer's intimate long-term knowledge about a specific athlete.

However, the above-described steps taken by a trainer may not suffice in all cases. In more severe cases, the athlete may have to see a physical therapist who in turn provides the required treatment based on the examination of the athlete.

Generally, such exercise and/or physical therapy recommendations are formulated relatively late after the onset of the athlete's injury. Also, these recommendations require significant hands-on work with the athlete, which can be both time consuming and expensive. Accordingly, there remains a need for cost effective systems and methods that can help with preventing or treating injuries with the athletes.

### SUMMARY

Inventive technology is directed to generating individualized recommendations for athlete's exercise (also referred to as a training) or physical therapy (also referred to as a service). In the context of this application, the term athlete encompasses professional and amateur athletes, as well as hobbyists, people who exercise, either regularly or on irregular basis, and others who engage in sports or exercise. All such categories of people (professional, amateur, consumers, etc.) are referred to as “athletes” in this application for simplicity and brevity.

In some embodiments, the athlete's uniform or other exercise clothing may be equipped with suitable sensors and/or data acquisition controllers that collect and interpret muscle activity data (e.g., muscle amplitude and frequency, heart rate, etc.). Such sensors may measure electrical impulses of the muscles representing muscle activity data. Collected data may be algorithmically processed to indicate muscle amplitude and/or frequency for one or more muscle groups of the user. In some embodiments, such algorithmic processing may include artificial intelligence and/or machine learning.

In some embodiments, individualized recommendations for athlete's exercise or physical therapy are based on measured differences between particular groups of muscles. For example, amplitudes of athlete's left and right quads are normally similar during a given exercise. However, if the athlete is excessively fatigued or injured, then the measured differences in the muscle amplitude may exceed a certain threshold, therefore indicating an excessive fatigue or an injury. As another nonlimiting example, the system may measure a difference between the amplitude of the athlete's hamstring and gluts. If the difference exceeds a predetermined threshold, the system concludes that the athlete is fatigued.

Based on these measured differences or anomalies in the muscle amplitude, the system may recommend, for example, a set of exercises or a particular physical therapy treatment (a particular service). For example, if the right hamstring is not recording a proper output, the inventive methods and systems advise the athlete to undergo a set of exercises. If the fatigue or injured seems of a more severe nature, the inventive methods and systems may recommend a physical therapist from the eco-system to connect with the user remotely. Collectively, such recommendations for exercise and/or physical therapy are herein referred to as a treatment recommendation. In many embodiments, such an early and rapid treatment recommendation may protect the athlete from further deteriorating into fatigue or injury, while being significantly more cost effective than conventional methods where the athlete is first evaluated by an expert person.

In one embodiment, a method for treating a fatigue or an injury of an athlete, the method includes: monitoring a first amplitude of a first muscle of the athlete by a first wearable muscle response sensor carried by the athlete; monitoring a second amplitude of a second muscle of the athlete by a second wearable muscle response sensor carried by the athlete; determining a difference between the first amplitude and the second amplitude; comparing the difference to a predetermined amplitude threshold; and based on the comparing, providing a treatment recommendation to the athlete.

In one aspect, the predetermined amplitude threshold is a first predetermined amplitude threshold. The method also includes comparing the difference to a predetermined second amplitude threshold. If the difference is greater than the first predetermined amplitude threshold and less than the second predetermined amplitude threshold, the method includes providing the treatment recommendation that is a prescribed exercise by an exercise database. If the difference is higher than a second predetermined amplitude threshold, the method includes providing the treatment recommendation that is a prescribed physical therapy by a physical therapy database.

In one aspect, if the difference is greater than the first predetermined amplitude threshold and less than the second predetermined amplitude threshold, the method includes providing a recommendation for a trainer by a coach database. If the difference is greater than the second predetermined amplitude threshold, the method includes providing a recommendation for a physical therapist by a physical therapist database.

In one aspect, the first muscle is a right hamstring (RH) and the second muscle is left hamstring (LH), and the predetermined amplitude threshold is expressed as:

$$Δ = \frac{R H - L H}{R H + L H} .$$

In another aspect, the predetermined amplitude threshold is 20%, 25%, 30%, 40%, 50%, or 60%.

In one aspect, the first muscle is a left hamstring (LH) and the second muscle is left glute (LG), and the predetermined amplitude is expressed as:

$$Δ = \frac{LH - LG}{LH + LG} .$$

In one aspect, the first wearable muscle response sensor is a wearable electromyography (EMG) sensor carried by the athlete.

In one aspect, the wearable EMG sensor is attached to a clothing of the athlete.

In one aspect, the method also includes: monitoring an orientation state (OS) of the athlete by a wearable orientation sensor carried by the athlete; and monitoring an activity state (AS) of the athlete by a wearable activity sensor carried by the athlete.

In one aspect, the wearable orientation sensor is a gyroscope and the wearable activity sensor is an accelerometer.

In one embodiment, a system for treating a fatigue or an injury of an athlete includes: a first wearable muscle response sensor configured for monitoring a first amplitude of a first muscle of the athlete; a second wearable muscle response sensor configured for monitoring a second amplitude of a second muscle of the athlete; a muscle activity tracker configured for receiving data from the first and second wearable muscle response sensors and for determining difference between the first amplitude and the second amplitude; and at least one database comprising recommendations for treating the fatigue or injury of the athlete in response to the determined difference between the first amplitude and the second amplitude.

In one aspect, the at least one database is configured for providing: a first treatment recommendation that is a prescribed exercise if the determined difference in the first amplitude and the second amplitudes is greater than a first predetermined amplitude threshold and less than a second predetermined amplitude threshold; and a second treatment recommendation that is a prescribed physical therapy if the difference in the first amplitude and the second amplitudes is greater than the second predetermined amplitude threshold.

In one aspect, the at least one database is further configured for providing: a recommendation for a trainer if the determined difference in the first amplitude and the second amplitudes is greater than the first predetermined amplitude threshold and less than the second predetermined amplitude threshold; and a recommendation for a physical therapist if the determined difference in the first amplitude and the second amplitudes is higher than the second predetermined amplitude threshold, providing a recommendation for a physical therapist.

In one aspect, the system includes a wearable controller attached to the athlete's clothing, the controller being configured to produce real-time or near real-time data based on input from the at least one wearable muscle response sensor.

In one aspect, the controller includes a wireless transceiver configured to communicate with the muscle activity tracker.

In one aspect, by the wearable muscle response sensor is a wearable electromyography (EMG) sensor carried by the athlete.

In one aspect, the system also includes a wearable orientation sensor configured for monitoring an orientation state (OS) of the athlete by; and a wearable activity sensor configured for monitoring an activity state (AS) of the athlete.

### BRIEF DESCRIPTION OF THE DRAWINGS

The foregoing aspects and many of the attendant advantages of this invention will become more readily appreciated with reference to the following detailed description, when taken in conjunction with the accompanying drawings, where:

FIG. 1 is a diagram illustrating an analytics system configured in accordance with an embodiment of the present technology.

FIG. 2 is a diagram illustrating components of a system in accordance with an embodiment of the presently disclosed technology.

FIG. 3 is block diagram illustrating components of a system in accordance with an embodiment of the presently disclosed technology.

FIGS. 4A and 4B are diagrams showing a measurement system in accordance with embodiments of the presently disclosed technology.

FIGS. 5-7 are graphs of muscle amplitude versus time for two different groups of muscles in accordance with embodiments of the presently disclosed technology.

FIGS. 8 and 9 are flowcharts of algorithmic exercise and physical therapy treatment recommendations in accordance with embodiments of the presently disclosed technology.

### DETAILED DESCRIPTION

System Overview

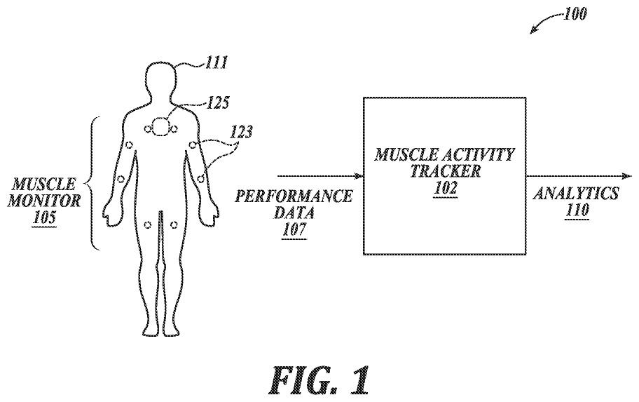

FIG. 1 is a diagram illustrating an analytics system 100 configured in accordance with an embodiment of the present technology. The system 100 includes a muscle activity tracker sub-system 102 (“muscle activity tracker 102”) and a muscle monitoring sub-system 105 (“muscle monitor 105”) that is worn by a user, such as an athlete or a user 111. The muscle monitor 105 may include an on-board controller 125 (“controller 125”) and sensors 123 that can be integrated into the athlete's clothing (not shown), such as the athlete's shirt, pants, etc. The athlete's clothing and the integrated controller 125 and sensors 123 may be collectively referred to as “smart compression clothing.” In operation, the controller 125 is configured to produce real-time or near real-time performance data (“real-time data”) 107 during an exercise, live game, practice session, or conditioning. Analytics 110 includes muscle response (MR) data, like frequency and amplitude activity for different groups of muscles. In different embodiments, analytics 110 may include data related to orientation state (OS) of the user, acceleration of the user, activity state (AS) of the user, etc. The analytics 110 may be produced over an evaluation period of a certain duration (e.g., 1 hour, 30 minutes, 15 minutes, 5 minutes, etc.). As described below, the system 100 can use the analytics 110 to produce indications, warnings, and alarms that alert the user or the trainer when an athlete is fatigued or injured.

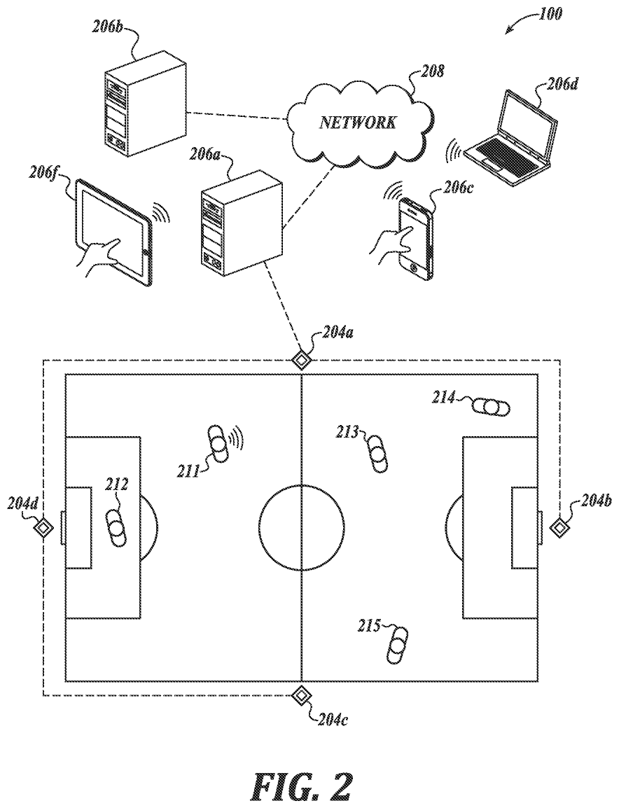

FIG. 2 is a diagram illustrating components of the system 100 in further detail. The system 100 illustrates interactions with multiple athletes, however, in other embodiments, the system may be focused on a single athlete. Furthermore, in different embodiments, the system 100 may include a subset of the illustrated components or additional components to those that are illustrated.

The muscle monitor 105 shown in FIG. 1 may be configured to communicate with one or more computing devices 206 via a plurality of gateway devices 204 positioned along monitoring region 227, such as a soccer-field, an athletic arena, gym, etc. The computing devices 206 are connected to one another via a network 208. The computing devices 206 are configured to receive, view, evaluate, store, and/or otherwise interact with data associated with the analytics 110 (FIG. 1B). For example, intermediary or back-end server devices 206a and 206b can exchange and process communications over the network 208, store a central copy of data, globally update content, etc. Examples of well-known computing devices, systems, environments, and/or configurations that may be suitable for use with the technology include, but are not limited to, personal computers, server computers, handheld or laptop devices 206d, mobile telephones 206c, tablet devices 206f, multiprocessor systems, microprocessor-based systems, set-top boxes, programmable consumer electronics, network PCs, minicomputers, mainframe computers, databases, distributed computing environments that include any of the above systems or devices, or the like.

One or more computing devices 206 can be configured to individually or collectively carry out the functions of the performance tracker 102 (FIG. 1) for producing the analytics 110. In various embodiments, the various computing devices 206 can process real-time data produced by one or more athletes 211, 212, 213, 214, 215 that are monitored in the monitoring region 227 of the gateways 204. As described below, the gateways 204 (e.g., gateways 204a, 204b, 204c, 204d) are configured to forward the real-time data 107 (FIG. 1) to the upstream computing devices 206 for processing.

Computing Devices

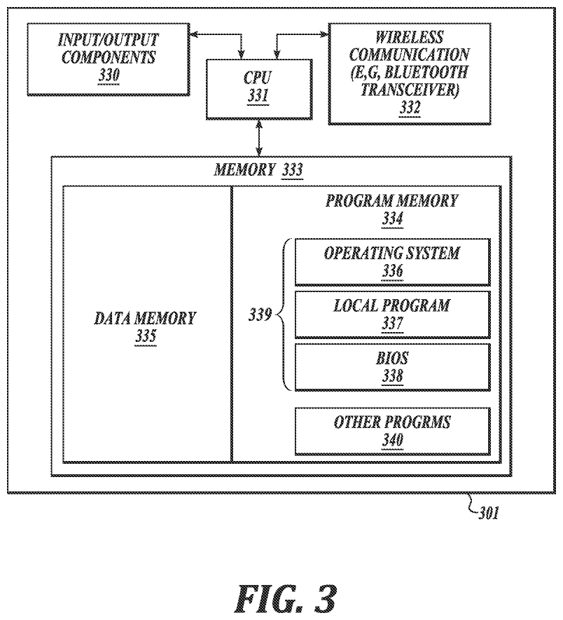

FIG. 3 is block diagram illustrating components that can be incorporated into a computing device 301, such as one of the computing devices 206 (FIG. 2), the gateways 204 (FIG. 2), and the muscle monitor 105 (FIG. 1A). The computing device 301 includes input and output components 330. Input components can be configured to provide input to a processor such as CPU 331, notifying it of actions. The actions are typically mediated by a hardware controller that communicates according to one or more communication protocols. The input components 330 can include, for example, a mouse, a keyboard, a touchscreen, an infrared sensor, a touchpad, a pointer device, a camera- or image-based input device, a pointer, and/or a microphone.

The CPU 331 can be a single processing unit or multiple processing units in a device or distributed across multiple devices. The CPU 331 can be coupled to other hardware components via, e.g., a bus, such as a PCI bus or SCSI bus. Other hardware components can include communication components 333, such as a wireless transceiver (e.g., a WiFi or Bluetooth transceiver) and/or a network card. Such communication components 332 can enable communication over wired or wireless (e.g., point-to point) connections with other devices. A network card can enable the computing device 301 to communicate over the network 208 (FIG. 2) using, e.g., TCP/IP protocols. Additional hardware components may include other input/output components, including a display, a video card, audio card, USB, firewire, or other external components or devices, such as a camera, printer, thumb drive, disk drive, Blu-Ray device, and/or speakers.

The CPU 331 can have access to a memory 333. The memory 333 includes volatile and non-volatile components which may be writable or read-only. For example, the memory can comprise CPU registers, random access memory (RAM), read-only memory (ROM), and writable non-volatile memory, such as flash memory, hard drives, floppy disks, CDs, DVDs, magnetic storage devices, tape drives, device buffers, and so forth. The memory 333 stores programs and software in programming memory 334 and associated data (e.g., configuration data, settings, user options or preferences, etc.) in data memory 335. The programming memory 334 contains an operating system 336, local programs 337, and a basic input output system (BIOS) 338, all of which can be referred to collectively as general software 339. The operating system can include, for example, Microsoft Windows™, Apple iOS, Apple OS X, Linux, Android, and the like. The programming memory 334 also contains other programs and software 340 configured to perform various operations. The various programs and software can be configured to process the real-time data 107 of the athlete 111 (FIG. 1) and produce corresponding analytics, such as during the live session 51, as described in greater detail below. Those skilled in the art will appreciate that the components illustrated in the diagrams described above, and in each of the diagrams discussed below, may be altered in a variety of ways.

Clothing and Sensors

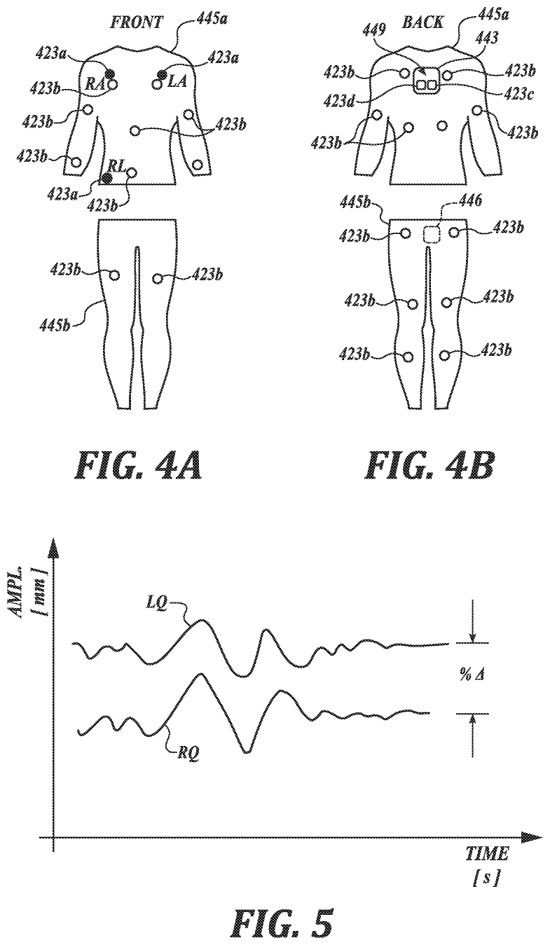

FIGS. 4A and 4B are diagrams showing a measurement system in accordance with embodiments of the presently disclosed technology. Referring to FIG. 4A, the controller 125 can be embedded within the athlete's clothing, such as a shirt 445a and pants 445b (collectively “clothing 445”). In other embodiments, the controller 125 can be inserted into a pocket 443 in the user's clothing and/or attached using Velcro, snap, snap-fit buttons, zippers, etc. In some embodiments, the controller 125 can be removable from the clothing 445, such as for charging the controller 125. In other embodiments, the controller 125 can be permanently installed in the athlete's clothing 445.

Referring to FIGS. 4A and 4B together, the controller 125 is operably coupled to muscle response sensors 423b that may be distributed over different muscle groups (e.g., pectoralis major, rectus abdominis, quadriceps femoris, biceps, triceps, deltoids, gastrocnemius, hamstring, and latissimus dorsi). The muscle response sensors 423b provide a measurement of the muscle activity during exercise. Amplitude and frequency of user's muscle response may be forwarded to the controller 125, and further to the computing devices 206 for data processing and display. A non-limiting example of the muscle response sensors 423b is an electromyography (EMG) sensor. The EMG sensors 423b can also be coupled to floating ground near the athlete's waist or hip.

In some embodiments, the clothing 445 may also be equipped with electrocardiogram (ECG) sensors 423a, orientation sensors 423c (e.g., a gyroscope), and acceleration sensors (also referred to as an activity sensor) 423d, for example, an accelerometer. The sensors 423 can be connected to the controller 449 using thin, resilient flexible wires (not shown) and/or conductive thread (not shown) woven into the clothing 445. The gauge of the wire or thread can be selected to optimize signal integrity and/or reduce electrical impedance.

The sensors 423a and 423b can include dry-surface electrodes distributed throughout the athlete's clothing 445 and positioned to make necessary skin contact beneath the clothing along predetermined locations of the body. The fit of the clothing can be selected to be sufficiently tight to provide continuous skin contact with the individual sensors, allowing for accurate readings, while still maintaining a high-level of comfort, comparable to that of traditional compression fit shirts, pants, and similar clothing. In various embodiments, the clothing 445 can be made from compressive fit materials, such as polyester and other materials (e.g., Elastaine) for increased comfort and functionality. In some embodiments, the controller 125 and the sensors 423 can have sufficient durability and water-resistance so that they can be washed with the clothing 445 in a washing machine without causing damage. In these and other embodiments, the presence of the controller 125 and/or the sensors 423 within the clothing 445 may be virtually unnoticeable to the athlete. In one aspect of the technology, the sensors 423 can be positioned on the athlete's body without the use of tight and awkward fitting sensor bands. In the context of this application, the sensors 423 and the controller 125 are referred to as “wearable” components. In general, traditional sensor bands are typically uncomfortable for an athlete, and athletes can be reluctant to wear them.

In additional or alternate embodiments, the muscle monitor 105 (FIG. 1) can include a separate controller 446 worn on the athlete's pants 445b. The separate controller 446 can be similar to the controller 125 worn on the athlete's shirt 445a, and is connected to the individual sensors 423 located on the pants 445b. The separate controller 446 can be configured to communicate with the controller 125 and/or with the gateways 204 (FIG. 2).

Controller Communication

In operation, the controller 125 of the muscle monitor 105 (FIG. 1) is configured to process and packetize the data it receives from the sensors 423 (e.g., the muscle response sensors 423b). The controller 125 (or controllers 446 or 449 shown in FIG. 4B) may broadcast the packetized data for detection by the gateway devices 204, which, in turn, forward the data to the performance tracker 105 (FIG. 1A) to produce required analytics (e.g., frequency and amplitude of muscle activity).

Muscle Activity Indication

FIGS. 5-7 are graphs of muscle amplitude versus time for two groups of muscles in accordance with embodiments of the presently disclosed technology. In each graph, the horizontal axis represents time (e.g., in seconds) and the vertical axis represents muscle amplitude in units of displacement (e.g., in mm). The illustrated graphs represent time series of the amplitude activity for particular muscle groups that was measured continuously by, for example, muscle response sensors 423b.

FIG. 5 illustrates muscle amplitude measurements for right quad (RQ) and left quad (LQ). The two muscle amplitudes retain a generally constant difference A, indicating a possible issue with athlete's performance (e.g., an injury). However, the difference A does not increase over time, as indicated by a constant difference between the two amplitudes, i.e., the two muscle amplitudes generally move in sync, albeit having different magnitudes. In some embodiments, the system 100 may make determinations as to whether the user needs attention by a physical therapist or a trainer based on the value of the difference A in the muscle amplitude of the RQ and LQ. In different embodiments, such threshold difference in the muscle amplitude may be normalized and expressed as:

$$Δ = \frac{L Q - R Q}{L Q + R Q} \qquad ( Eq . 1 )$$

In some embodiments, the system 100 may make determinations as to whether the user needs attention by a physical therapist or a trainer based on the value of difference A in the muscle amplitude of the RQ and LQ. For example, above a certain minimum value and up to a certain threshold value of Δ, the athlete is provided with a recommendation for exercise. When the value of Δ exceeds certain threshold value, the athlete may be referred to a physical therapy. Some non-limiting sample values of the threshold Δ are 20%, 25%, 30%, 40%, 50%, or 60%.

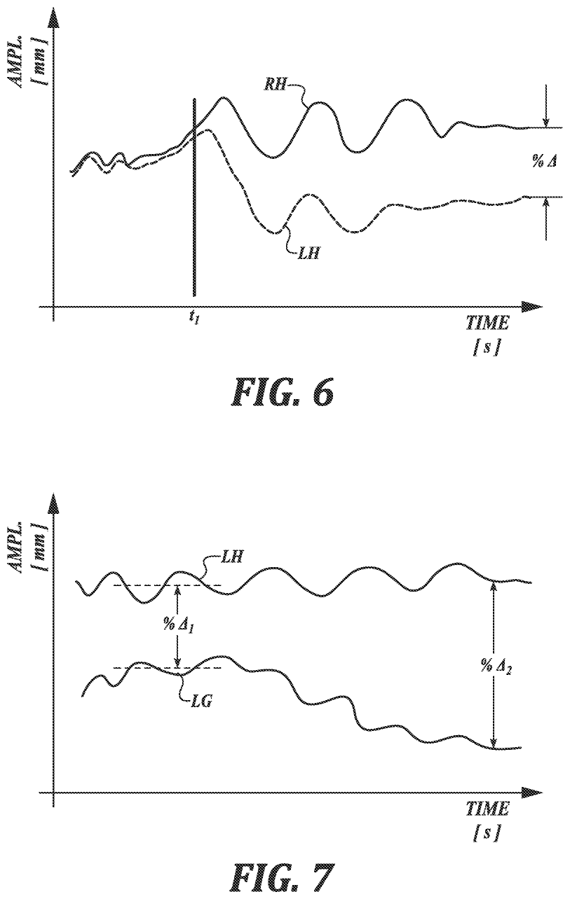

FIG. 6 illustrates muscle amplitude measurements for right hamstring (RH, solid line) and left hamstring (LH, dash line). In the beginning of the exercise and up to the time ti, muscle amplitude for the RH and LH is generally comparable, increasing and decreasing with the intensity of exercise. As the user becomes more fatigued in the course of the exercise, the difference in the muscle amplitude between the RH and LH becomes more pronounced. As the difference in the muscle amplitude reaches a certain predetermined value, the user may be entering a dangerous zone, that is, a zone of excessive fatigue or an increased likelihood of injury. In different embodiments, such threshold difference in the muscle amplitude may be expressed as:

$$Δ = \frac{RH - LH}{RH + LH} \qquad ( Eq . 2 )$$

As explained above, different values of threshold Δ generally result in different recommendations.

FIG. 7 illustrates muscle amplitude measurements for left hamstring (LH) and left glute (LG). In the beginning of the exercise and up to the time ti, muscle amplitude for the LH and LG remains within limit of Δ₁, which may be an acceptable difference based on the difference in the type of muscle. As the user becomes more fatigued with the exercise, a difference in the muscle amplitude between the LH and LG becomes larger. For example, such difference may reach a value of Δ₂, indicating a zone of excessive fatigue or an increased likelihood of injury. In different embodiments, such threshold difference in the muscle amplitude may be expressed as:

$$Δ = \frac{LH - LG}{LH + LG} \qquad ( Eq . 3 )$$

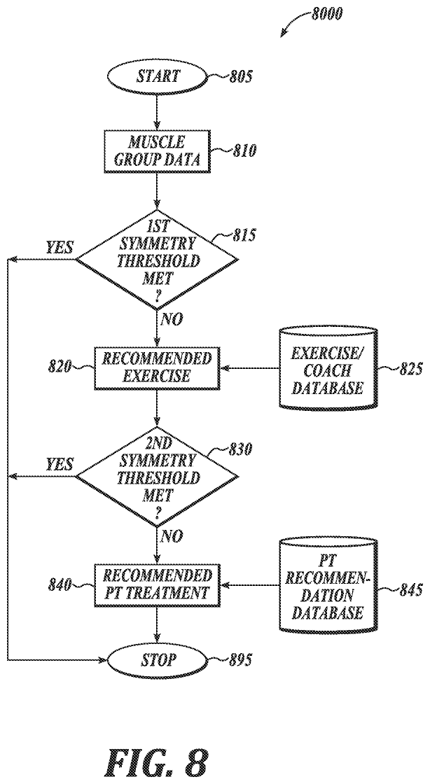

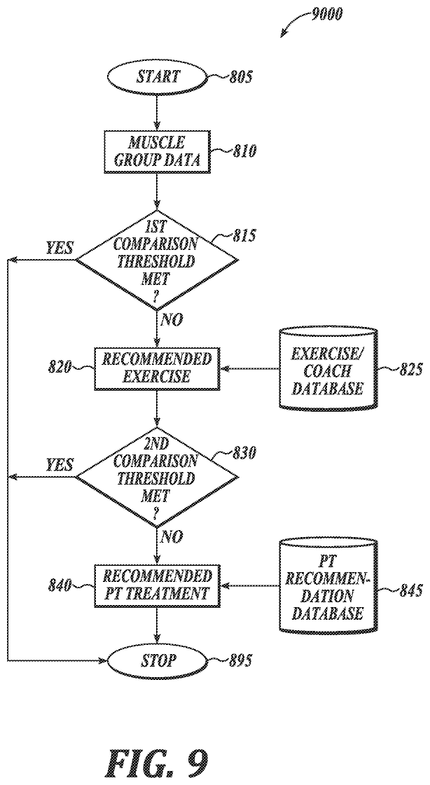

Some sample determinations of the exercise and physical therapy recommendations are described in more details with respect to FIGS. 8 and 9 below.

FIG. 8 is a flowchart of exercise and physical therapy treatment algorithmic recommendations in accordance with embodiments of the presently disclosed technology. In the illustrated embodiment, a comparison is made between the same or similar groups of muscles to establish whether symmetry is present between the groups of muscles. In some embodiments, the method 8000 may include only some of the steps in the flowchart, or may include additional steps that are not illustrated in the flowchart.

The method starts in block 805. In block 810, certain muscle groups are selected for observation. Some examples of such muscle groups are right quad (RQ) and left quad (LQ), right hamstring (RH) and left hamstring (LH), etc. In block 815, a determination is made as to whether a first symmetry threshold (e.g., Ai) is met, that is, whether a difference between the measured groups of muscles is below the first symmetry threshold. A nonlimiting example of such determination is provided in, for example, Equation 1. If the first symmetry threshold is met, the assumption is that the athlete is not fatigued or injured, and method may end in block 895. If the first symmetry threshold is not met, that is, a difference between the muscle amplitude of the two groups of muscles exceeds certain threshold, the system recommends an exercise to remedy the condition in block 820. Other algorithms may be used in different embodiments. In different embodiments, the algorithms may be based on artificial intelligence or machine learning. Such exercise may be recommended based on a database 825 that includes a listing of possible exercises and/or coaches (also referred to as an exercise database or a coach database).

In block 830, a determination is made as to whether a second symmetry threshold (e.g., Δ₂) is met, that is, whether a difference between the measured groups of muscles has reached the second symmetry threshold. In some embodiments, the second symmetry threshold indicates a condition that is more severe than the one related to the first symmetry threshold. A nonlimiting example of such determination is provided in, for example,

FIG. 7, indicating that a particular problem (fatigue or injury) deteriorated further with time. In some embodiments, not meeting the second symmetry threshold Δ₂ causes the method to proceed to block 840 where a physical therapy treatment is recommended to the athlete. The physical therapy and/or the therapists may be recommended based on data available in a database 845 (also referred to as a physical therapy or a physical therapist database). In different embodiments, the databases 825 and 845 may be the same databases or different databases. The method ends in block 895.

FIG. 9 is a flowchart of exercise and physical therapy treatment algorithmic recommendations in accordance with embodiments of the presently disclosed technology. In some embodiments, the method 9000 may include only some of the steps in the flowchart, or may include additional steps that are not illustrated in the flowchart. In the illustrated embodiment of method 9000, a comparison is made between dis-similar groups of muscles to establish whether the athlete is fatigued or injured. For example, it is known that for a well-rested and healthy athlete different muscle groups exhibit amplitudes that are in sync with each other. Stated differently, different muscle groups for such athlete (user) are characterized by the amplitudes which increase and decrease together as a group, but the difference in the amplitudes remains within certain thresholds Δ. In contrast, the difference in muscle amplitudes of these different muscle groups for a fatigued or injured athlete tend to exceed the prescribed thresholds Δ. A non-limiting example of such comparison of the amplitudes of different muscle groups is shown in FIG. 7. For example, the first comparison threshold Δ₁ (or other algorithmic threshold) in block 815 may trigger a recommendation for exercise/coach based on the database in block 825. Analogously, the second comparison threshold Δ₂ in block 830 may trigger a recommendation for physical therapy/physical therapist based on the database in block 845.

While various advantages associated with some embodiments of the disclosure have been described above, other embodiments may also exhibit such advantages, and not all embodiments need necessarily exhibit such advantages to fall within the scope of the invention. For example, while various embodiments are described in the context of an athlete (e.g., a professional or collegiate athlete), in some embodiments users of the system can include novice or intermediate users, such as users, trainers, and coaches associated with a high school sports team, an athletic center, a professional gym, physical therapist, etc. Accordingly, the disclosure can encompass other embodiments not expressly shown or described herein. In the context of this disclosure, the word “approximately” indicates a difference of +/−5% of the stated value.

What is claimed is:

1. A method for treating a fatigue or an injury of an athlete, the method comprising:
monitoring a first amplitude of a first muscle of the athlete by a first wearable muscle response sensor carried by the athlete;
monitoring a second amplitude of a second muscle of the athlete by a second wearable muscle response sensor carried by the athlete;
determining a difference between the first amplitude and the second amplitude;
comparing the difference to a predetermined amplitude threshold; and
based on the comparing, providing a treatment recommendation to the athlete.

2. The method of claim 1, wherein the predetermined amplitude threshold is a first predetermined amplitude threshold, the method further comprising:
comparing the difference to a predetermined second amplitude threshold;
if the difference is greater than the first predetermined amplitude threshold and less than the second predetermined amplitude threshold, providing the treatment recommendation that is a prescribed exercise by an exercise database; and
if the difference is higher than a second predetermined amplitude threshold, providing the treatment recommendation that is a prescribed physical therapy by a physical therapy database.

3. The method of claim 2, further comprising:
if the difference is greater than the first predetermined amplitude threshold and less than the second predetermined amplitude threshold, providing a recommendation for a trainer by a coach database; and
if the difference is greater than the second predetermined amplitude threshold, providing a recommendation for a physical therapist by a physical therapist database.

4. The method of claim 1, wherein the first muscle is a right hamstring (RH) and the second muscle is left hamstring (LH), and wherein the predetermined amplitude threshold is expressed as:
$$Δ = \frac{R H - L H}{R H + L H} .$$

5. The method of claim 2, wherein the predetermined amplitude threshold is 20%, 25%, 30%, 40%, 50%, or 60%.

6. The method of claim 1, wherein the first muscle is a left hamstring (LH) and the second muscle is left glute (LG), and wherein the predetermined amplitude is expressed as:
$$Δ = \frac{LH - LG}{LH + LG} .$$

7. The method of claim 2, wherein by the first wearable muscle response sensor is a wearable electromyography (EMG) sensor carried by the athlete.

8. The method of claim 7, wherein the wearable EMG sensor is attached to a clothing of the athlete.

9. The method of claim 1, further comprising:
monitoring an orientation state (OS) of the athlete by a wearable orientation sensor carried by the athlete; and
monitoring an activity state (AS) of the athlete by a wearable activity sensor carried by the athlete.

10. The method of claim 9, wherein the wearable orientation sensor is a gyroscope and the wearable activity sensor is an accelerometer.

11. A system for treating a fatigue or an injury of an athlete, comprising:
a first wearable muscle response sensor configured for monitoring a first amplitude of a first muscle of the athlete;
a second wearable muscle response sensor configured for monitoring a second amplitude of a second muscle of the athlete;
a muscle activity tracker configured for receiving data from the first and second wearable muscle response sensors and for determining difference between the first amplitude and the second amplitude; and
at least one database comprising recommendations for treating the fatigue or injury of the athlete in response to the determined difference between the first amplitude and the second amplitude.

12. The system of claim 11, wherein the at least one database is configured for providing:
a first treatment recommendation that is a prescribed exercise if the determined difference in the first amplitude and the second amplitude is greater than a first predetermined amplitude threshold and less than a second predetermined amplitude threshold; and
a second treatment recommendation that is a prescribed physical therapy if the difference in the first amplitude and the second amplitude is greater than the second predetermined amplitude threshold.

13. The system of claim 12, wherein the at least one database is further configured for providing:
a recommendation for a trainer if the determined difference in the first amplitude and the second amplitude is greater than the first predetermined amplitude threshold and less than the second predetermined amplitude threshold; and
a recommendation for a physical therapist if the determined difference in the first amplitude and the second amplitude is higher than the second predetermined amplitude threshold, providing a recommendation for a physical therapist.

14. The system of claim 11, further comprising a wearable controller attached to the athlete's clothing, the controller being configured to produce real-time or near real-time data based on input from the at least one wearable muscle response sensor.

15. The system of claim 14, wherein the controller includes a wireless transceiver configured to communicate with the muscle activity tracker.

16. The system of claim 11, wherein by the wearable muscle response sensor is a wearable electromyography (EMG) sensor carried by the athlete.

17. The system of claim 12, further comprising:
a wearable orientation sensor configured for monitoring an orientation state (OS) of the athlete by; and
a wearable activity sensor configured for monitoring an activity state (AS) of the athlete.
